(19) United States
(12) Patent Application Publication
(10) Pub. No.: US 2008/0114367 A1
(43) Pub. Date: May 15, 2008

(54) Method and relaxable distracters for in-situ formation of intervertebral disc prosthesis

(75) Inventor: Thomas L. Meyer, New Albany, OH (US)

Correspondence Address: KREMBLAS, FOSTER, PHILLIPS & POLLICK, 7632 SLATE RIDGE BOULEVARD, REYNOLDSBURG, OH, 43068, US

(73) Assignee: Syberspine Limited

(21) Appl. No.: 11/595,582

(22) Filed: Nov. 10, 2006

(51) Int. Cl. A61B 17/58; A61M 29/00; A61F 2/44

(52) U.S. Cl. 606/90; 606/191; 623/17.16

(57) ABSTRACT

A method and a distracter for replacing a removed, intervertebral disc with a prosthesis. An inter-body distracter is inserted within the intervertebral space between adjacent vertebrae, the vertebrae are distracted and are supported by the distracter. A fluid, curable polymer is injected into the intervertebral space and around the distracter and then the polymer is cured to a semi-rigid, pliable, elastically deformable state. The distracter is then disabled from supporting compressive forces applied by the vertebrae and remains in place. The preferred distracter is a scissors jack having intermediate bearings joined by links to support feet. One of the bearings has a threaded lateral bore and the second bearing has a lateral bore. A rod extends through the bores and has a threaded portion extending from a first end of the rod and threadedly engaging the threaded bearing. The inside diameter of the second bore is greater than the outside diameter of the threaded portion of the rod so the rod is axially slidable through the second bore. A thrust bearing is fixed on the threaded rod and positioned laterally outwardly of the second bearing for forcing the second bearing laterally toward the first bearing upon rotation of the rod in one direction and for permitting the rod to be withdrawn from the bearing by rotating the rod in the opposite direction and disengaging it from the first bearing. Alternative distracters are also disclosed.

### BACKGROUND OF THE INVENTION

1. Field Of The Invention

This invention relates generally to surgical devices and procedures and more particularly relates to devices and a procedure for replacement of and/or augmentation of intervertebral disc nucleus in the spine.

2. Description Of The Related Art

The vertebral column or spine provides flexible mechanical support and comprises a series of vertebrae having adjacent vertebrae separated from each other by an intervertebral disc. Each disc consists of an annulus fibrosus forming an envelope surrounding an inner nucleus pulposus. The annulus fibrosus, while flexible, has a relatively small elasticity. The nucleus pulposus is a viscous fluid that, when contained in the annulus fibrosus, allows the disc to function as a semi-rigid, pliable, cushion. The disc allows relative articulation of adjacent vertebrae and elastically supports axial forces.

Disc degeneration commonly occurs as a result of aging or injury. The annulus fibrosus gets weaker or becomes herniated and the pulposus may be extruded through it. Consequently, the disc loses some or all of its load supporting and articulation capabilities, the load forces may become distributed improperly on the vertebrae and the nucleus pulposus may press against nerve roots causing pain.

One form of treatment is for a surgeon to fuse together or otherwise immobilize the adjacent vertebrae on opposite sides of the damaged disc. More recently, surgeons have sought functional restoration by replacing a disc, or a part of a disc, with a prosthetic replacement. Many of these prosthetic devices utilize a flexible polymer because polymers have been identified that both have mechanical properties that simulate the physical characteristics of a disc and are biologically compatible.

Examples of artificial disc prostheses include the devices illustrated in U.S. Pat. Nos. 4,932,975, 6,375,682, 6,419,704 and 6,932,843, and U.S. published applications 2003/0004574 and 2004/0167626. One difficulty with many such devices is that they are complicated mechanical structures that are therefore expensive and they are difficult to properly install. Polymers have been proposed that can be used in such devices and are liquid when installed but are then cured to a semi-rigid, pliable, elastically deformable, gel-like state that simulates the mechanical properties of the nucleus pulposus or the entire disc.

However, a problem and complication with these disc replacement devices is that, in order to install the disc replacement, the adjacent vertebrae must be distracted to provide a properly sized intervertebral space for receipt of the disc replacement and the vertebrae must be held in their distracted position during insertion of the disc replacement. The distracter is therefore a rigid, though adjustable, device that permits the surgeon to accurately position the adjacent vertebrae and maintain the vertebrae in the position they were placed by the surgeon. However, the presence of a distracter in the same intervertebral space where the disc replacement is to be located interferes with insertion of the disc replacement. The distracter also needs to be removed after disc replacement is installed because the distracter is a rigid device applying forces to the adjacent vertebrae that would negate the desired elastic and flexible properties of a disc replacement.

It is therefore an object and feature of the invention to provide distracters and a method for forming a disc replacement in-situ that overcomes these problems while simplifying the procedure and the disc replacement structure.

### BRIEF SUMMARY OF THE INVENTION

The method of the invention is to distract adjacent vertebrae from which the interposed and damaged nuclear material can been removed, leaving the annulus fibrosis intact, inject a curable polymer into the disc space around the distracter and against the vertebrae with the distracter still in position distracting the vertebrae, cure the polymer to a semi-rigid, pliable, elastically deformable state so the cured polymer functionally replaces a spinal disc, and then relax the distracter by disabling the distracter from supporting compressive forces applied by the vertebrae so that the distracter remains in place but becomes free to move within the cured polymer and not interfere with the compression, flexing and load supporting function of the cured polymer.

A distracter that can be relaxed and disabled is similar to known scissors jacks because it has at least two, longitudinally opposite support feet, at least two pairs of laterally spaced links, each pair of links being pivotally joined together at their proximal ends by a bearing and having their respective distal ends each pivotally joined to one of the feet. Also like a conventional scissor jack, a first one of the bearings has a threaded lateral bore and the second bearing has a lateral bore, though preferably smooth. A rod, having threads extending from a first end, is threadedly engaged in the threaded bore and extends through the second bore and beyond by a distance to permit manual rotation of the rod by a surgeon. However, unlike a conventional scissors jack, the second one of the bearings has an inside diameter that is larger than the outside diameter of the threaded portion of the rod so that the rod can be slid axially out of the jack when the rod is rotated sufficiently to disengage the threads of the rod from the threaded bore in the first bearing. A thrust bearing is fixed on the threaded rod in a location spaced from the first end of the rod and is positioned laterally outwardly of the second bearing. Consequently, the thrust bearing will seat against and force the second bearing laterally toward the first bearing upon rotation of the rod in a direction that moves the rod axially into the first bearing. Alternative distracters include: (1) an inflatable balloon-like envelope that distracts the vertebrae upon inflation with a fluid and relaxes by exhausting the fluid; (2) a distracter that has a support leg or legs formed of a shape memory metal alloy or polymer that is formed at its high temperature to a relaxed configuration of the distracter, then mechanically deformed to an elongated configuration for retaining the vertebrae in distraction and, after the curable polymer prosthesis is cured, heated to return to its relaxed configuration; and (3) a distracter that has two support feet forced apart by a key extending between the feet and relaxed by removal of the key.

### BRIEF DESCRIPTION OF THE SEVERAL VIEWS OF THE DRAWINGS

FIG. 1 is a top plan view of a distracter embodying the invention.

FIG. 2 is a view in side elevation of the embodiment illustrated in FIG. 1.

FIG. 3 is a right side view of the embodiment illustrated in FIG. 1.

FIG. 4 is a bottom view of the embodiment illustrated in FIG. 1.

FIG. 5 is an enlarged view in vertical section taken substantially along the line 5-5 of FIG. 1 of a portion of the embodiment illustrated in FIG. 1 and illustrating in more detail the relationship of the rod, bearings and thrust bearing.

FIG. 6 is a diagrammatic view illustrating the placement of the distracter of FIGS. 1-5 in the intervertebral space in accordance with the method of the invention.

FIG. 7 is a diagrammatic view illustrating the injection of a curable polymer around the distracter in accordance with the method of the invention and after the procedure illustrated in FIG. 6.

FIG. 8 is a diagrammatic view illustrating the relaxed, disabled distracter in place within the cured polymer after the threaded rod is removed following the procedure illustrated in FIG. 7.

FIG. 9 is a view in perspective of an alternative embodiment of the invention which is an enclosed, flexible, balloon-like envelope with a fluid inlet/outlet.

FIG. 10 is a top view of the embodiment illustrated in FIG. 9.

FIG. 11 is a view in side elevation and showing a quarter vertical section of the embodiment illustrated in FIG. 9.

FIG. 12 is a view in side elevation of the embodiment illustrated in FIG. 9 in a relaxed state.

FIG. 13 is a view in side elevation of the embodiment illustrated in FIG. 9 positioned in an intervertebral space.

FIG. 14 is a view in side elevation of another alternative embodiment of the invention formed of a shape memory metal alloy or polymer and in a relaxed state.

FIG. 15 is a view in side elevation of the embodiment of FIG. 14 in an extended, distracting state.

FIG. 16 is a view in side elevation of the embodiment of FIG. 14 positioned in an intervertebral space.

FIG. 17 is a view in front elevation of yet another alternative embodiment of the invention and having a pair of spaced support feet forced apart by a central key.

FIG. 18 is a view in side elevation of the embodiment of FIG. 17.

FIG. 19 is a top plan view of the embodiment of FIG. 17.

FIG. 20 is a top plan view illustrating the addition of a swing keel as an optional improvement of the embodiment of FIGS. 1-5.

FIG. 21 is a side sectional view, taken substantially along the line 21-21 of FIG. 20, of the swing keel embodiment of FIG. 20 showing the swing keel structure in a retracted, undeployed position.

FIG. 22 is a side sectional view, taken substantially along the line 21-21 of FIG. 20, of the swing keel embodiment of FIG. 20 but showing the swing keel structure in a deployed position.

FIG. 23 is a side view of an alternative pivot link that may be substituted for links illustrated in FIGS. 20-22.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose.

### DETAILED DESCRIPTION OF THE INVENTION

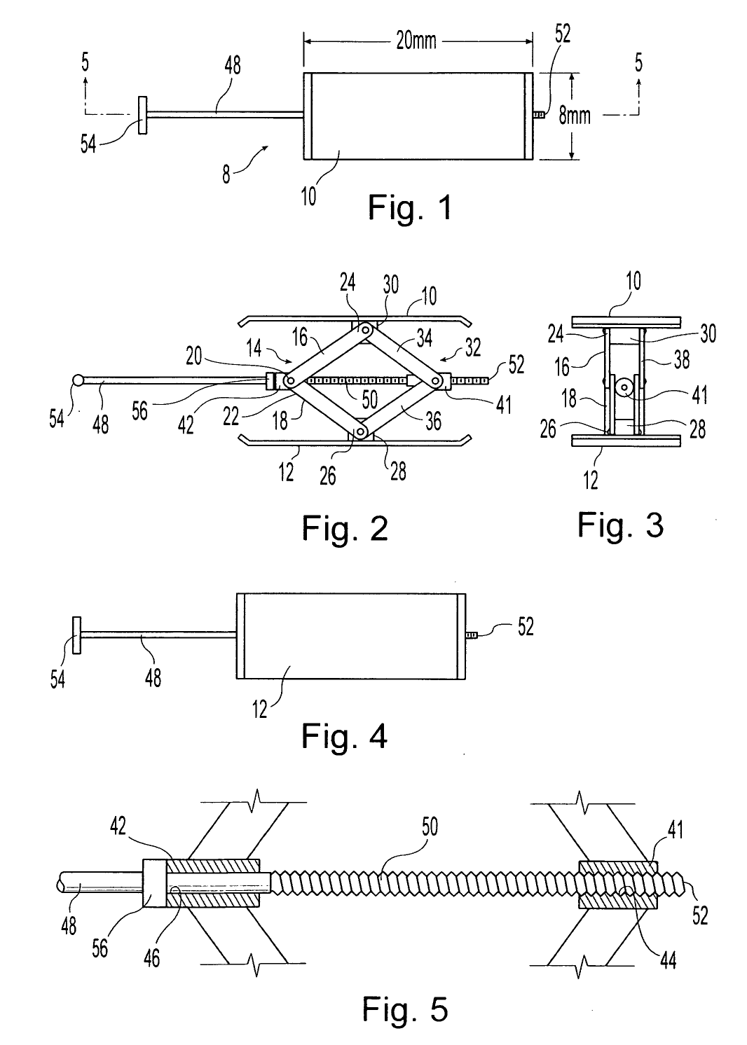

FIGS. 1-5 illustrate an inter-body distracter 8 embodying the invention and capable of use in practicing the method of the invention. Two, longitudinally opposite, support feet 10 and 12 face longitudinally outwardly for engaging the surface of the adjacent bodies that are to be distracted. In the method of the invention those bodies are vertebrae but the distracter can be used in other environments. A first pair 14 of links 16 and 18 are pivotally joined together at their proximal, engaging ends 20 and 22. Their respective distal ends 24 and 26 are each pivotally joined to a different one of the feet 10 and 12 at respective foot bearings 28 and 30 that are fixed to the support feet 10 and 12. A second, mirrored pair 32 of links 34 and 36 are laterally spaced from the first pair 14 of links 16 and 18.

On the opposite side of the foot bearings 28 and 30 are two more laterally spaced pairs of links that are pivotally connected together and to the feet 10 and 12 in the same manner. Although two pairs of links can be used, the two additional pairs of links provide additional stabilization of the relative orientation of the support feet 10 and 12.

A pair of laterally spaced bearings 41 and 42 are interposed between the support feet 10 and 12 and are pivotally connected to the links. Each bearing is pivotally joined to the proximal, engaging ends of a different one of the pairs of links, such as the proximal ends 20 and 22 pivotally joined to the bearing 42. A first one of the bearings, bearing 41, has a threaded lateral bore 44. The second one of said bearings, bearing 42, has a second lateral bore 46.

A rod 48 is formed with a threaded portion 50 extending from a first end 52 of the rod 48. The threaded portion 50 threadedly engages the threaded lateral bearing 41. The rod 48 extends laterally through the second bore 46 of the second bearing 42 and further extends beyond the bearing 42 by a distance that permits manual rotation of the rod. Preferably, a cross bar 54 is fixed to the end of the rod 48 to facilitate grasping and rotating the rod 48. Importantly, the inside diameter of the second bore 46 is greater than the outside diameter of the threaded portion 50 of the rod 48. This dimensional relationship makes the rod axially slidable through the second bore.

A thrust bearing 56 is fixed on the threaded rod 48, spaced from the first end 52 of the rod 48 and positioned laterally outwardly of the second bearing 42. The thrust bearing 56 seats against the second bearing 42 and forces the second bearing 42 laterally toward the first bearing 41 upon rotation of the rod in the direction that drives the rod 48 axially into the first bearing 41. Additionally, because of the diameter relationships described above, the rod 48 can be rotated in the opposite direction so it is driven axially out of the first bearing 41. Then, upon disengagement of the rod 48 from the first bearing 41, the rod can be withdrawn through the second bearing 42 and removed from the distracter.

This removal of the rod 48 disables the distracter from supporting compressive forces applied by the vertebrae. Therefore the distracter becomes relaxed or limp and provides no force resistance if its support feet are moved toward or away from each other by the vertebrae. Consequently, the distracter can initially function as a distracter as needed by the surgeon and then the surgeon can remove the rod causing to distracter to become limp and non-functional so it applies no forces to any object if left in the intervertebral space.

An inter-body distracter embodying the invention may be manufactured of presently known or future materials used for surgical implants or other medical devices. For example, these include PEEK, titanium or stainless steel.

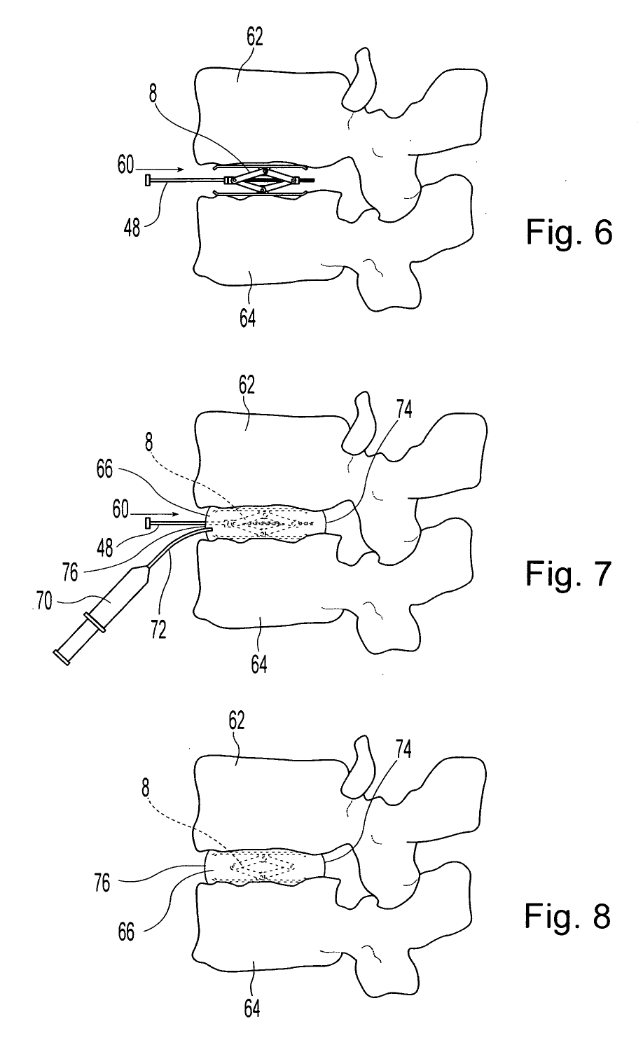

The manner of using the distracter for replacing a removed, intervertebral disc with a disc prosthesis in accordance with the invention is illustrated in FIGS. 6 through 8. Currently known or future surgical approaches for spinal surgery may be used, such as lateral, anterior, anterolateral, extreme lateral, posterior, posterolateral. Either open or minimally invasive types of surgery may be performed and accomplished through ports, cannulas or traditional incisions.

Referring first to FIG. 6, after any damaged disc is removed from the intervertebral space 60, the distracter 8 is inserted into the intervertebral space 60 between adjacent vertebrae 62 and 64 and its support feet are deployed apart to engage and distract the vertebrae 62 and 64 to positions determined by the surgeon. The distraction is accomplished by rotating the lateral rod 48 which is preferably aligned in the midsagittal plane and extends posteriorly. The direction of rotation depends upon whether the mating screw threads of the rod 48 and the first bearing 41 are conventional right hand threads or the opposite. The surgeon rotates the rod 48 in the direction that drives the rod further into the first bearing 41 which causes the thrust bearing 56 to be axially translated toward the first bearing 41. As the opposite pairs of links are moved toward each other, the support feet 10 and 12 are forced apart.

Referring to FIG. 7, after the surgeon is satisfied with the positioning of the vertebrae 62 and 64, a fluid, curable polymer 66 is extruded from its container, such as a cannulated syringe 70 through a flexible tube 72, and injected into the intervertebral space 60 and around and within the distracter 8. The fluid polymer 66 flows against the interfacing surfaces of the vertebrae 62 and 64 and also flows anteriorly and posteriorly to form an anterior boundary 74 and posterior boundary 76 at positions selected by the surgeon.

After the surgeon is satisfied with the distribution of the polymer 66 in the intervertebral space 60 and the positions of the boundaries 74 and 76, the polymer is cured to a semi-rigid, pliable, elastically deformable state capable of supporting compressive forces applied to it by the vertebrae. The curing is performed in the manner described in the prior art for example by irradiation, such as by UV radiation, thermal (body temperature or other local environmental stimulus) or other externally applied energy source.

After curing is completed to the state desired by the surgeon, the distracter is disabled from supporting compressive forces applied by the vertebrae. In the distracter described above, this is done by rotating the rod in the direction opposite to the direction for deploying the distracter, unthreading it from the first bearing 41 and then withdrawing it posteriorly along the axis of the rod 48. As illustrated in FIG. 8, this leaves the distracter in situ along with the cured polymer. Consequently, the distracter 8 supports the vertebrae in distraction only temporarily until the polymer is cured. After the distracter is disabled, it becomes dynamic; that is, able to move freely without applying any force on the vertebrae. Instead, the vertebrae are supported by the cured polymer, which forms a pad that functions as a replacement disc.

The invention is not the composition of the polymer. The polymer needs to be biologically compatible, able to flow before curing so it can be injected into the intervertebral space and within and around the distracter. It also needs to be curable to a semi-rigid, pliable, elastically deformable, gel-like state in which it retains a nearly constant volume when compressed. When cured, its molecules need to be able to move with respect to each other like a liquid, but the molecules need to have sufficient molecular attraction to also provide elasticity. It needs to be stiff or viscous enough to support spinal loading forces applied by vertebrae in the spinal column.

There are various polymers that exhibit these characteristics that have been used for surgery or are being investigated and proposed. One such polymer that appears particularly suitable for practicing the invention is a protein hydrogel that has been described in the prior art literature. Such materials include BioDisc by Cryolife, Atlanta, Ga. and NuCore by Spinewave, Irvine, Calif.

The method of the invention is not confined to the preferred distracter that has been described. There are various alternative distracters that can be inserted and then, after the curable polymer cures, relaxed and disabled from applying a force to separate the vertebrae so that it can remain in situ.

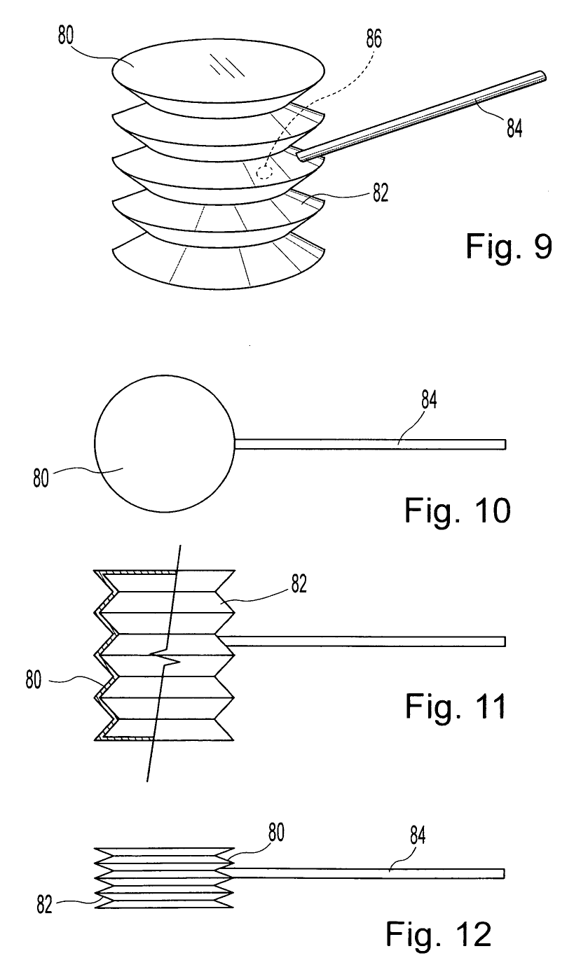

FIGS. 9-13 illustrate an alternative distracter that has an enclosed, flexible, balloon-like envelope 80 that is inflatable with a fluid. The preferred envelope 80 is formed in a cylindrical bellows configuration with pleats 82. Because the envelope 80 will be inflated with a fluid so that it expands to distract adjoining vertebrae, it is necessary that it expand in at least one direction. The bellows configuration advantageously provides an envelope that expands principally in a longitudinal direction, for distracting the vertebrae, with minimal expansion in the lateral direction. In order to perform the distraction function, it is necessary that the envelope be expandable by inflation at least to a longitudinal length substantially equal to the vertebral spacing of normal human vertebrae. Although this dimension varies among individuals, it is believed that the envelope should be expandable by inflation to about 20 mm. The envelope must also be deflatable to a size that will fit into an intervertebral space after the surgeon has removed some or all of the damaged disc but before final distraction of the discs. Although this necessary size varies with individuals and particular cases, it is believed that the envelope should be collapsible to about 7 mm. Additionally, in order to allow space for receipt of a sufficient quantity of curable polymer to form an effective disc prosthesis, the envelope 80 should not, after inflation, have a lateral width any greater than substantially one third the lateral width of a human vertebra.

The envelope must also have an inflation/deflation inlet through which the envelope can be inflated and later deflated. The preferred inflation/deflation inlet comprises a tube 84 that extends from the envelope 80 and is in fluid communication with the interior of the envelope. It preferably intersects the envelope 80 at its sidewall so that it does not become constricted by being pinched between one of the longitudinally opposite ends of the envelope and one of the vertebrae. Alternatively, the inflation/deflation inlet can be an opening 86 through the envelope and having an interior valve adjacent the opening 86 like those commonly used on basketballs and other inflatable objects. Such valves are openable for inflation by penetration through the opening and valve of a fluid conveying needle. However, when the needle is withdrawn, the valve operates as a check valve that closes to seal the opening as a result of fluid pressure within the envelope.

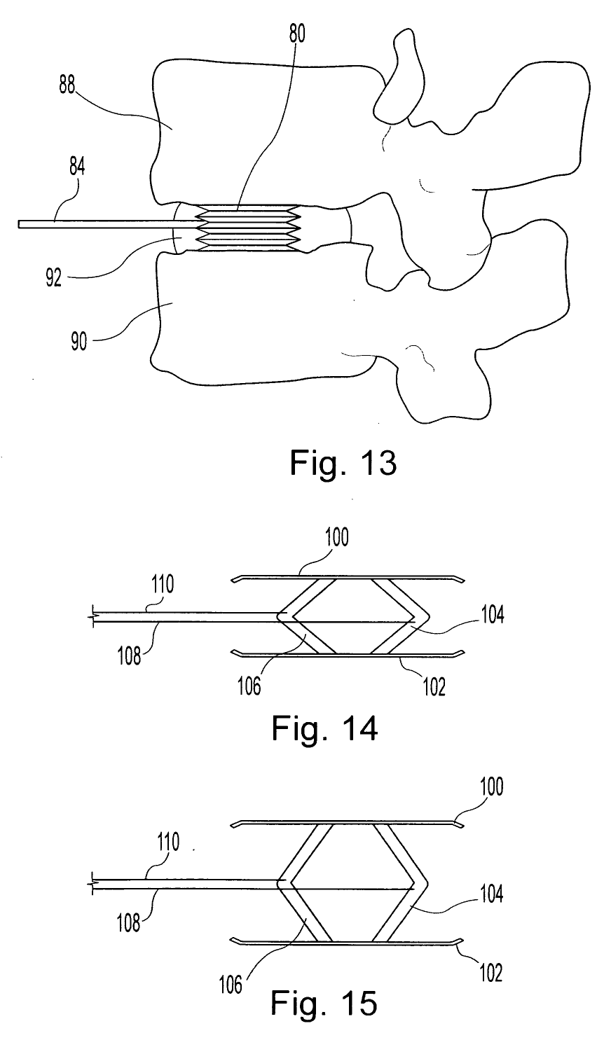

The operation of the embodiment of FIGS. 9-13 is illustrated in FIG. 13. The inflatable envelope 80, in a deflated, collapsed state, is inserted by the surgeon between the vertebrae 88 and 90. A fluid is then forced under pressure through the tube 84 into the envelope 80. When the envelope has forced the vertebrae 88 and 90 to the spacing judged appropriate by the surgeon, the inflow of fluid is stopped. The fluid pressure is then held constant, for example by shutting a valve in the fluid supply system or clamping the tube 84. Preferably, the fluid is a liquid, such as a saline solution, because a liquid is nearly incompressible. Therefore, a liquid will more rigidly hold the vertebrae in the distracted state. A curable polymer 92, of the type previously described, is then injected into the intervertebral space around and against the envelope 80 and cured as described in connection with the previously described embodiments. After curing, the portion of the tube 84 that protrudes from the cured polymer 92 may be cut off. That allows the fluid to escape from the interior of the envelope 80 thereby relaxing the envelope so it is disabled from applying a separation force against the vertebrae. If the inflation/deflation inlet is the alternative opening and valve structure described above, after the polymer is cured, a fluid conveying needle, configured like those used to inflate basketballs, is then inserted through the cured polymer and into the inflation/deflation inlet opening to drain the inflation fluid.

FIGS. 14-16 illustrate another alternative embodiment of a distracter that is useful in the method of the present invention. It utilizes a property of materials known in the prior art as shape memory metal alloys or shape memory polymers. As known in that art, a shape memory material, for example NiTi, is a material that remembers its geometry. A memory metal alloy is formed in a particular initial configuration at a first, sufficiently high temperature and then cooled. After cooling, it can be mechanically deformed into other configurations and, as long as its temperature does not exceed the first temperature, upon being heated to a sufficient temperature, it will return to its initial configuration. Shape memory polymers operate in an analogous manner.

FIGS. 14 and 15 illustrate a distracter utilizing this shape memory property. As with the embodiment of FIGS. 1-8, the distracter of FIGS. 14 and 15 has at least two, longitudinally opposite support feet 100 and 102. Support legs 104 and 106 extend between and are connected to the support feet 100 and 102. These support legs 104 and 106 are constructed of the shape memory metal alloy or polymer. The support legs can apply oppositely directed longitudinal forces against the support feet 100 and 102 to retain adjoining vertebrae in a distracted position. Each support leg has a high temperature shape that it “remembers” at which each support leg spaces the support feet apart by a distance that is less than the vertebral spacing of normal human vertebrae. This is its relaxed state. Each support leg is mechanically deformed from its remembered configuration to a distracting configuration that spaces the support feet 100 and 102 farther apart than the spacing of the remembered configuration. More specifically, the support legs are deformed to space the support feet apart by a distance that is substantially equal to the desired vertebral spacing of normal human vertebrae.

A pair of electrical conductors 108 and 110 are connected to the distracter for applying an electrical current through each support leg. This allows an electrical current to be applied through the support legs for heating them by resistance heating and cause the support legs to return towards their high temperature remembered configuration so the legs will relax the distracter and cease applying a longitudinal force upon the support feet.

There are a wide variety of support leg configurations that can be used and different quantities of such legs may also be employed. The illustrated support legs each consist of two angled bar segments joined together at a knee. Since their ends are fixed to the support feet, they can be easily mechanically deformed in their cooled state to force the support feet apart by applying equal and opposite forces on the knees, such as with surgical pliers, to force the knees closer together and thereby spread the support feet further apart. The support legs can also be formed in an arcuate configuration and the support feet spread apart in the same manner. Although two support legs are illustrated, for greater strength, four or more such support legs can be used. For example, they can be arranged at the corners of a rectangle. A support leg in a helical configuration can also be used, in which case, only one would be necessary.

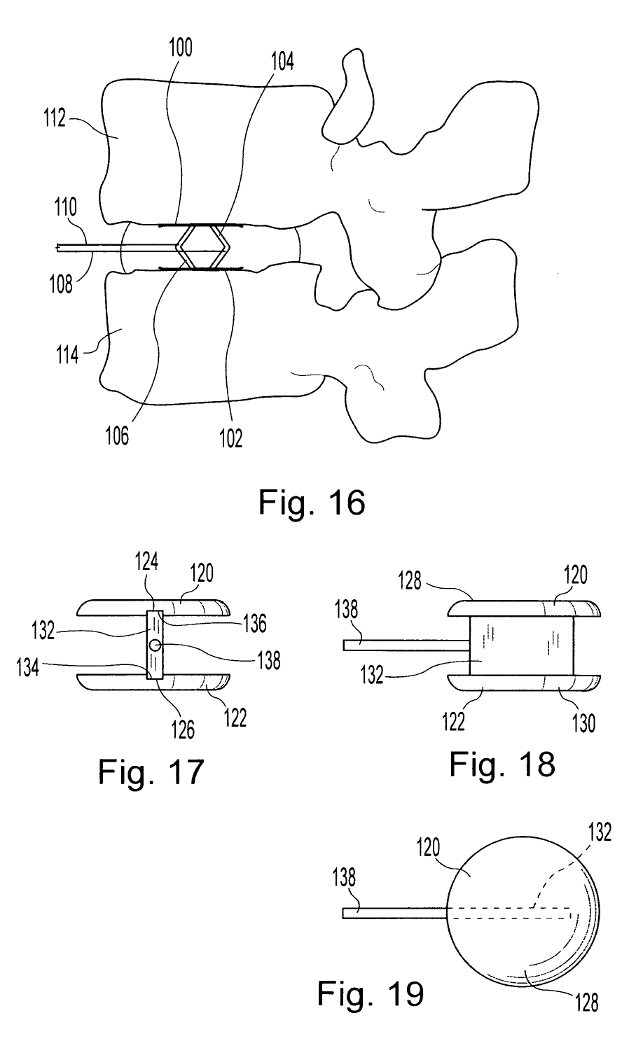

The operation of the embodiment illustrated in FIGS. 14 and 15 is illustrated in FIG. 16. After the surgeon has removed part or all of the damaged disc, the surgeon has two options for distracting the vertebrae 112 and 114. In one option, before insertion of the distracter of the invention, the support legs are deformed to space the support feet apart by the desired spacing between the vertebrae 112 and 114. This can be done either by the surgeon or by a manufacturer who may supply distracters in a variety of sizes. The vertebrae are distracted with a conventional distracter to the desired spacing and the distracter of the invention is inserted in position between the vertebrae 112 and 114. The conventional distracter is then removed and the distracted state is maintained by the distracter of the invention. Using the second option, the surgeon positions the distracter of the invention between the vertebrae with the distracter in its relaxed, remembered configuration. The surgeon then deforms the support legs to force the support feet apart and distract the vertebrae to the desired spacing. Using either option, the wires 108 and 110 are oriented to extend out of the intervertebral space. After inserting the distracter and performing the distraction according to one of the options, the surgeon them extrudes a curable polymer between the vertebrae and around the distracter and cures it. After curing, an electrical power source is connected to the wires 108 and 110 to heat the support legs and thereby relax the distracter toward its remembered configuration.

FIGS. 17-19 illustrate yet another distracter that can be used for performing the method of the present invention. The distracter has a pair of longitudinally opposite support feet 120 and 122. Each support foot has a guide channel 124 and 126 on one side and a support surface 128 and 130 on the opposite surface for seating against adjacent vertebrae. The guide channel can be formed in various ways, such as a diametrical slot in each support foot as illustrated, or with two parallel series of protruding posts or bosses. A key 132 has opposite edges 134 and 136 slidable in and along the guide channels 124 and 126 of the support feet 120 and 122. The opposite edges 134 and 136 are spaced apart a distance to separate the support surfaces 128 and 130 of support feet 120 and 122 by a distance that is substantially equal to the vertebral spacing of normal human vertebrae.

In order to facilitate insertion and removal of the key 132, a key removal bar 138 extends from the key 132 substantially in the plane of the opposite edges 124 and 126 and in a direction approximately parallel to those edges. The surgeon is able to grasp the key removal bar for sliding the key into the channel and also for sliding the key out of the channel. The key removal bar 138 can be integrally formed with the key 132 or it can have a male threaded end that is threadedly engaged to a female threaded hole in an edge of the key 132. The slot engaging edges 134 and 136 of the key 132 can be tapered to diverge closer to the key removal bar 138 with the slots 124 and 126 of the support feet 120 and 122 having corresponding, mating tapers. With this alternative configuration, the opposite slot engaging edges 134 and 136 form a wedge that forces the support feet 120 and 122 apart as the key 132 is slid into the slots 124 and 126. This can be used to distract the adjacent vertebrae.

In the operation of the embodiment of FIGS. 17-19, the surgeon can either (1) initially distract the vertebrae with a conventional distracter, insert the assembled distracter of the invention and then remove the conventional distracter or (2) insert the support feet and use a tapered key and slot configuration to distract the vertebrae by sliding the tapered key into the slot. The curable polymer is then extruded into the intervertebral space and around the distracter of the invention and then cured. After curing, the key 132 is slid posteriorly through the polymer and out from between the support feet. Because the key does not need to be very wide, it can easily make a clean cut through any cured polymer along its path of removal.

Distracters of each embodiment of the invention must have dimensions so that they are able to be inserted between vertebrae, do not extend out beyond the boundaries of the vertebrae, are capable of distracting the vertebrae a distance chosen by a surgeon and so that they allow sufficient remaining volume between the vertebrae for insertion of the curable polymer. Because vertebrae and their intervertebral spaces vary in size, different distracters may be constructed in different sizes in order to optimize their dimensions for the location in which they are intended to be used. Taking into account that the distracters embodying the invention my may be applied to the cervical, thoracic and lumbar regions of the spine, typical ranges of dimensions for the distracters would be:

anterior-posterior length: 6 mm to 40 mm

medio-lateral width: 2 mm to 16 mm

cranio-caudal height: 4 mm to 20 mm.

The aspect ratio of these dimensions can also be varied. Consequently, it is not necessary that a distracter having one dimension in a particular place within the dimension range, such as nearer the larger end of the range, have all its dimensions in the same place within all the ranges.

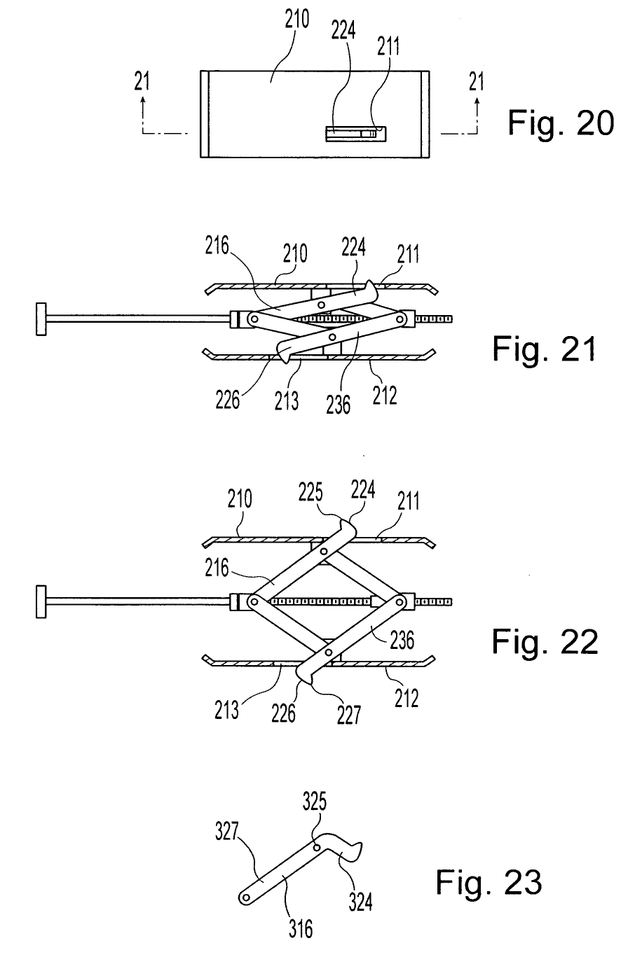

FIGS. 20-28 illustrate an improvement of the embodiment of FIGS. 1-8. Referring first to FIGS. 1-3, there may be a risk that, when the rod 48 is rotated to force the bearings 41 and 42 toward each other in order to move the support feet 10 and 12 apart during distraction of the vertebrae, the distracter may also rotate. This could happen as a result of the torque that is applied to the rod 48 by the surgeon being transmitted through the frictionally engaged rod 48 and bearing 41 and/or bearing 42. If the transmitted torque is sufficient, one or both of the support feet 10 and 12 may slide along the surface of the vertebrae and become misaligned. The torque might even cause the entire distracter to rotate 90°. One solution would be to provide a high friction surface or an array of small protrusions on the support feet 10 and 12. However, because the distracter feet remain in position against the vertebrae after the polymer is cured, the surgeon may feel that the roughened surface is undesirable.

FIGS. 20-23 illustrate an additional structural modification for preventing rotation and misalignment of the distracter as a result of the torque applied to the rod 48 (FIG. 2). Referring to FIGS. 20-22, the two opposite support feet 210 and 212 have rectangular slots 211 and 213 to form an aperture entirely through each of the support feet 210 and 212. The distal ends 224 and 226 of at least two opposite links 216 and 236 (the ends that are pivotally joined to each of the support feet) extend distally beyond their pivotal connection to the support feet to form swing keels 224 and 226 that swing through each of the opposite support feet as the distracter is extended to distract adjacent vertebrae. To permit this, the two rectangular slots 211 and 213 are positioned in alignment with the path of the swing keels 224 and 226. The slots 211 and 213 are made sufficiently large to permit passage of the swing keels through the slots and deployment into engagement with adjacent vertebrae. Each swing keel 224 and 226 is desirably formed with a sharp tooth 225 and 227 that protrudes in approximately opposite cranio-caudal directions, when the distracter is operatively positioned, in order to lock into the adjacent vertebrae. The swing keels 224 and 226 need to protrude beyond the support feet only a few millimeters into the vertebrae when the distracter is spread to its maximum distraction position. This structure not only locks the support feet to the vertebrae but also allows the swing keels to retract out of engagement with the vertebrae when the distracter is relaxed after the polymer is cured.

FIG. 23 illustrates a link 316 that may be substituted for each of the links 216 and 236. The link 316 differs from the links 216 and 236 by the orientation of its swing keel extension. The swing keel 324 extends from the pivot 325 at an angle that is not 1800 opposite to the direction of the link arm 327. This modification allows a further extension of the distracter before the swing keel 324 is deployed through a slot into engagement with the vertebrae, if that is desired. The angle of the extension may be more or less than the angle illustrated in FIG. 23.

While certain preferred embodiments of the present invention have been disclosed in detail, it is to be understood that various modifications may be adopted without departing from the spirit of the invention or scope of the following claims.

1. A method for replacing a removed, partially removed, or retained but structurally deficient, intervertebral disc with a prosthesis, the method comprising:
(a) inserting an inter-body distracter within the intervertebral disc space between adjacent vertebrae, distracting the vertebrae and temporarily supporting the vertebrae in distraction with the distracter;
(b) injecting a fluid, curable polymer into the intervertebral space and around the distracter;
(c) curing the polymer to a semi-rigid, pliable, elastically deformable state capable of supporting compressive forces applied by the vertebrae; and
(d) disabling the distracter from supporting compressive forces applied by the vertebrae.

2. A method in accordance with claim 1 wherein the intervertebral space is injected with a polymer that is a protein hydrogel.

3. A method in accordance with claim 1 wherein an inter-body distracter is inserted that comprises (a) at least two, longitudinally opposite support feet; (b) at least two pairs of laterally spaced links, each pair of links being pivotally joined together at proximal, engaging ends and having their respective distal ends each pivotally joined to one of said feet; (c) a pair of bearings, each bearing being pivotally joined to proximal, engaging ends of a different one of the pair of links, a first one of said bearings having a threaded lateral bore and the second one of said bearings having a second lateral bore; (d) a rod having a threaded portion extending from a first end of the rod and threadedly engaging the threaded lateral bearing, the rod extending laterally through the second bore and beyond by a distance to permit manual rotation of the rod, the inside diameter of the second bore being greater than the outside diameter of the threaded portion of the rod so the rod is axially slidable through the second bore; and (e) a thrust bearing fixed on the threaded rod, the thrust bearing being spaced from said first end of the rod and positioned laterally outwardly of the second bearing for forcing the second bearing laterally toward the first bearing upon rotation of the rod in one direction and for permitting the rod to be withdrawn from the bearing upon rotation of the rod in the opposite direction and disengagement of the rod from the first bearing.

4. A method in accordance with claim 3 wherein the intervertebral space is injected with a polymer that is a protein hydrogel.

5. A method in accordance with claim 1, and more particularly comprising, prior to inserting the inter-body distracter, removing interposed and damaged nuclear material from the intervertebral disc and leaving the annulus fibrosis intact.

6. An intervertebral distracter comprising:
(a) at least two, longitudinally opposite support feet;
(b) at least two pairs of laterally spaced links, each pair of links being pivotally joined together at proximal, engaging ends and having their respective distal ends each pivotally joined to one of said feet;
(c) a pair of bearings, each bearing being pivotally joined to proximal, engaging ends of a different one of the pair of links, a first one of said bearings having a threaded lateral bore and the second one of said bearings having a second lateral bore;
(d) a rod having a threaded portion extending from a first end of the rod and threadedly engaging the threaded lateral bearing, the rod extending laterally through the second bore and beyond by a distance to permit manual rotation of the rod, the inside diameter of the second bore being greater than the outside diameter of the threaded portion of the rod so the rod is axially slidable through the second bore; and
a thrust bearing fixed on the threaded rod, the thrust bearing being spaced from said first end of the rod and positioned laterally outwardly of the second bearing for forcing the second bearing laterally toward the first bearing upon rotation of the rod in one direction and for permitting the rod to be withdrawn from the bearing upon rotation of the rod in the opposite direction and disengagement of the rod from the first bearing.

7. An intervertebral distracter in accordance with claim 6 wherein
(a) there are four pairs of laterally spaced links, each pair of links being pivotally joined together at proximal, engaging ends and having their respective distal ends each pivotally joined to one of said feet; and
(b) each of said pair of bearings is pivotally joined to proximal, engaging ends of a different two of the four pairs of links.

8. An intervertebral distracter in accordance with claim 6 wherein
(a) the distal ends of at least two of the links that are pivotally joined to each of the support feet extend distally beyond their pivotal connection to the support feet to form swing keels that swing through each of the opposite support feet when the distracter is extended to distract adjacent vertebrae; and
(b) at least two, longitudinally opposite support feet have slots in alignment with the swing keels, the slots being positioned and sufficiently large to permit passage of the swing keels through the slots and into engagement with adjacent vertebrae.

9. An intervertebral distracter comprising:
an enclosed, flexible, balloon-like envelope that is inflatable with a fluid in at least a longitudinal direction and having an inflation/deflation inlet, the envelope being inflatable to a lateral width no greater than substantially one third the lateral width of a human vertebra and to a longitudinal length substantially equal to the vertebral spacing of normal human vertebrae.

10. An intervertebral distracter in accordance with claim 9 wherein the inflation/deflation inlet comprises a tube in fluid communication with the interior of the envelope.

11. An intervertebral distracter in accordance with claim 9 wherein the inflation/deflation inlet comprises an opening through the envelope having a valve that is openable by penetration of the opening and valve by a fluid conveying needle and is a check valve upon withdrawal of the needle to close from fluid pressure within the envelope.

12. An intervertebral distracter comprising:
(a) at least two, longitudinally opposite support feet;
(b) at least one support leg extending between and connected to the support feet and constructed of a shape memory metal alloy or polymer, each support leg having a high temperature shape at which each support leg spaces the support feet apart by a distance that is less than the vertebral spacing of normal human vertebrae, each support leg being mechanically deformed to space the support feet apart by a distance that is substantially equal to the vertebral spacing of normal human vertebrae; and
(c) a pair of electrical conductors connected to the distracter for applying an electrical current through each support leg for heating each support leg and causing it to return towards its high temperature shape and cease applying a longitudinal force upon the support feet.

13. An intervertebral distracter comprising:
(a) at least two, longitudinally opposite support feet, each support foot having a guide channel on one side and a support surface on the opposite for seating against a vertebra; and
(b) a key having opposite edges slidable in the guide channels of the support feet, the opposite edges being spaced apart a distance to separate the support feet by a distance that is substantially equal to the vertebral spacing of normal human vertebrae.

14. An intervertebral distracter in accordance with claim 10 and further comprising a key removal bar extending from the key substantially in the plane of the opposite edges and in a direction approximately parallel to the edges for sliding the key out of the channels.
